(19) United States
(12) Patent Application Publication
(10) Pub. No.: US 2004/0029205 A1
(43) Pub. Date: Feb. 12, 2004

(54) Diagnostic system for differentiating sputum from saliva

(76) Inventors: Parker Adams Small JR., Gainesville, FL (US); Darla J. Gaskins, Gainesville, FL (US); Kenneth H. Rand, Gainesville, FL (US); Shih-Wen Huang, Gainesville, FL (US)

Correspondence Address: BEUSSE BROWNLEE WOLTER MORA & MAIRE, 390 N. ORANGE AVENUE, SUITE 2500, ORLANDO, FL, 32801, US

(21) Appl. No.: 10/209,396

(22) Filed: Jul. 30, 2002

(51) Int. Cl. C12Q 1/44; C12Q 1/37

(52) U.S. Cl. 435/19; 435/23

(57) ABSTRACT

A novel diagnostic system for rapidly, non-invasively and inexpensively differentiating sputum from saliva. The disclosed methods utilize a test strip or stick assay, or other similar assays, for detecting the presence of leukocyte esterase which correlates to the presence of sputum in a patient sample.

### FIELD OF THE INVENTION

The present invention relates to a novel diagnostic system for differentiating a sputum sample from saliva sample. Once the sample is determined to include a sufficient amount of sputa, the sample is further analyzed to determine the presence of respiratory pathogens associated with pulmonary diseases or conditions.

### BACKGROUND OF THE INVENTION

It is common for patients afflicted with a respiratory infection to suffer from coughing, fever, chest pain, and shortness of breath. A patient suffering from such symptoms will often seek the advice of a clinician in an effort to minimize or overcome their discomfort. Given the host of respiratory pathogens, and overlap in symptoms caused thereby, identifying the particalur pathogen responsible for the infection can be a challenge for the clinician. The chance of mis-diagnosis is significant, which can be costly and potentially dangerous for the patient. For example, should a clinician mis-diagnose the particular respiratory infection, the clinician could prescribe a series of antibiotics not designed for treatment of the pathogen involved. As a result, the infection can worsen unabatedly. In the case of more severe respiratory infections, such as pneumonia, the condition can become critical.

In view of the foregoing problem, methods have been developed to assist the clinician in diagnosing respiratory infections. Typically, a physician obtains a sample of sputa from a patient complaining of discomfort and showing signs of a pulmonary disease or condition. Sputum is matter is matter produced in respiratory tract. Sputum comes in a variety of colors and forms. Fresh sputum originating from the lungs should contain pathogens if the patient is suffering from a pulmonary infection or condition. Sputum generally accumulates in the lungs as a result from the progression of such an infection. The following list identifies commonly known variations of sputum.

| | |
|---|---|
| Sputum aeroginosum | green sputum |
| Albuminoid sputum | yellow frothy sputum of persons |
| | from whom large amounts of pleural |
| | fluid have been withdrawn, believed |
| | to be due to pulmonary edema |
| Sputum coctum | the opaque mucopus of the later |
| | stages of bronchitis and laryngitis |
| Sputum crudum | clear tenacious mucus of the early |
| | stages of laryngitis and bronchitis |
| Sputum cruentum | bloody sputum |
| Globular sputum | yellow spherical lumps and |
| | characteristic of the late stages of |
| | tuberculosis |
| Green sputum | stained with a green pigment as in |
| | certain cases ofjaundice |
| Icteric sputum | stained with a greenish or yellow tint |
| | by bile pigments as in jaundice |
| Mossagate sputum | grayish, opalescent, gelatinous |
| | mottled sputum, usually projected |
| | from the mouth in a more or less |
| | globular form during coughing |
| | which is characteristic of diseases of |
| | the trachea |
| Nummular sputum | shaped as rounded disks like a coin |
| Prune juice sputum | dark red-dish brown, bloody sputum |
| | of certain forms of pneumonia, |
| | cancer of the lung, gangrene, etc. |
| Rusty sputum | stained with blood or blood pigments |
| | as seen in pneumonia |

Typically, after the sputa sample is collected, the physician sends the sample to a clinical laboratory for analysis to determine if there are any possible bacterial pathogens within the sample to isolate the specific pulmonary disease and/or condition.

Unfortunately, between 10-40% of the samples acquired from a patient in need of treatment thereof, contain only saliva and not a significant amount of sputum to test for bacterial pathogens associated with pulmonary diseases or conditions. Since the analysis of saliva samples without sputa provides no useful information to the physician about the pathogenic processes in the lungs or nasal cavity, both wasted work and delay in patient care/treatment results in approximately 10-40% of the sputa samples submitted for analysis.

This is especially troublesome in doctor's offices or hospital settings where patients are submitting a vast number of unsatisfactory sputa samples over a period of time which are sent to clinical laboratories. Once clinical laboratories receive the sputa samples they are typically analyzed by plating the sputa on blood agar, then making and reading the gram stain. The gram stain is examined under the microscope, and if it contains many buccal epithelial cells and few leucocytes, it is judged to be saliva and therefore reported unsatisfactory. There are at least two major problems associated with the submission of unsatisfactory sputa sample to clinical laboratories for analysis for pulmonary. First, there are costs associated with unsatisfactory sputa samples to both the medical industry and the patient. Clinical laboratories may charge hospitals and physicians for the analysis of unsatisfactory samples, but also may charge for repeated tests until a subsequent satisfactory sample has been obtained. In addition, the repeat procurement and testing of samples contributes to increased workloads, which in turn, creates extra costs to support an increased workforce.

Secondly, there are many problems associated with patient care. For example, there can be delays in patient care caused by repeated sputa samples needed to be obtained every 24 hours due to unsatisfactory samples being collected by the technicians and sent to clinical laboratory for analysis. Furthermore, patient care suffers when antibiotics are mis-prescribed to patients that may not require antibiotics or patients in need of antibiotics and are not treated due to unsatisfactory samples being obtained by technicians not containing the necessary sputum in the sample which would reveal the presence of a specific bacterial pathogen.

Until now, no rapid, inexpensive differential diagnostic method exists to differentiate sputum from saliva in order for the physician to obtain a satisfactory sputa sample containing a sufficient amount of sputum. The satisfactory sputa sample may then be sent to a clinical laboratory to determine through further analysis whether respiratory pathogens associated with pulmonary diseases or conditions are present.

Many test strip or stick assays are used to measure proteolytic enzyme activity in bodily fluids such as urine, blood, nasal secretions, and saliva; however, none of these tests are designed to differentiate sputum from saliva in order to diagnose whether a patient is in need of treatment for a pulmonary disease or condition. See for example, Eggelston, et al., Mediators of Immediate Hypersensitivity in Nasal Secretions during Natural Colds and Rhinovirus Infection Acta Otolaryngol. 1984; suppl. 413:25-35; Baumgarten, et al, Plasma Kallikrein During Experimentally Induced Allergic Rhinitis: Role in Kinin Formation and Contribution to TAME-Esterase Activity in Nasal Secretions, J. Immunol. 1986; 137:977-982; Wang et al., Correlations between Complaints, Inflammatory Cells and Mediator Concentrations in Nasal Secretions after Nasal Allergen Challenge and during Natural Allergen Exposure, Int. Arch. Allergy Immunology 1995; 106:278-285; Sigurs et al., Eosinophil cationic protein in nasal secretion and in serum and myeloperoxidase in serum in respiratory syncytial virus bronchiolitis: relation to asthma and atopy, Acta Paediatr 1994; 83:1151-5; Okuda et al., A Novel Method of Counting Eosinophils in Nasal Secretion of Allergic Rhinitis by Hemocytometric Method, Int. Arch. Allergy Immunol. 1994; 104 (suppl. 1):6; Kowalski et al., Neutrophil chemotactic activity (NCA) in nasal secretions from atopic and nonatopic subjects, Allergy 1993; 48:409-414; Igarashi et al., Analysis of nasal secretions during experimental rhinovirus upper respiratory infections, J. Allergy Clin. Immunol. 1993; 92:722-731; Florman, et al., Rapid Non-invasive Techniques for Determining Etiology of Bronchitis and Pneumonia in Infants and Children, Clin. Chest Med. 1987; 8:669-679; U.S. Pat. Nos. 4,657,855; and 4,278,763.

None of the patents or publications described above teach or suggest a test device on bodily fluids such as saliva to differentiate sputum from saliva. In addition, none of the references teach or suggest the application of a test strip or stick device to differentiate sputum from saliva for detecting bacteria pathogens associated with pulmonary diseases and conditions.

U.S. Pat. No. 5,051,358 issued on Sep. 24, 1991 to Jonathan J. Witt teaches a method for determining the presence of or evaluation of periodontal diseases in humans or lower animals. The patent teaches a method of contacting saliva from a human or animal, in which the saliva sample is diagnosed with a leukocyte esterase detection reagent to determine the amount of leukocyte esterase present in the saliva colorimetrically. No color change is an indication of good dental health and a color change is an indication of a periodontal disease. The method as taught in this patent does not differentiate sputum from saliva. From the disclosure, the samples obtained by the methods disclosed in this patent would not include a sufficient amount of sputum which is necessary for detecting any bacterial pathogens that would suggest pathologic processes taking place in the lungs.

There has been a long-felt need in the art for a rapid, inexpensive, non-invasive diagnostic system capable of differentiating between different types of bodily secretions, specifically a method for differentiating sputum and saliva to determining the presence of a specific bacterial pathogen associated with pulmonary diseases or conditions.

### BRIEF SUMMARY OF THE INVENTION

This invention provides a novel diagnostic system for rapid, non-invasive and inexpensive testing for differentiating sputa sample from saliva sample by the use of a dip stick or strip assay for detecting the presence or absence of certain markers, such as leukocyte esterase. The subject methods increase the efficiency in determining the presence of pathogens associated with a pulmonary disease or condition.

According to one aspect of the invention, at least one reagent test strip assay specifically adapted for rapidly, non-invasively and inexpensively detecting leukocyte esterase or protease activity is used to test sputa samples from a patient to differentiate sputa samples from saliva samples. When leukocyte esterase or protease activity is found within a sputa sample, this information is used to determine whether the sputa sample contains sputum. This indication allows for further analysis by clinical laboratory testing to identify the specific pathogen associated with a respiratory infection or condition. Once an accurate diagnosis has been achieved, the patient is treated with the appropriate medications, such as antibiotics specific to isolated bacterial pathogens found in the sputa sample.

Accordingly, it is an object of this invention to provide a rapid, non-invasive and inexpensive diagnostic system to determine the presence of bacterial pathogens associated with pulmonary diseases or conditions.

A further object of this invention is to provide a diagnostic system that reduces the delay in patient care caused by unsatisfactory sputa samples obtained from the patient.

It is still a further object of the present invention to provide a diagnostic system that can incorporate a wide variety of test strip or stick assays that detect the presence or absence of leukocyte esterase or protease in a test sample.

The foregoing has outlined some of the more pertinent objectives of the present invention. These objectives should be construed to be merely illustrative of some of the more prominent features and applications of the invention. Many other beneficial results can be attained by applying the disclosed invention in a different manner of modifying the invention as will be described.

It is to be understood that the foregoing general description and the following detailed description are exemplary and explanatory only and are not to be viewed as being restrictive of the present, as claimed. These and other objects, features and advantages of the present invention will become apparent after a review of the following detailed description of the disclosed embodiments and the appended claims.

### DETAILED DESCRIPTION OF THE INVENTION

The present invention relates to a novel diagnostic system for differentiating sputa samples from saliva samples to improve diagnostic techniques in determining the presence or absence of pulmonary diseases or conditions. In general, a diagnosis of pulmonary diseases or conditions is made by a combination of clinical symptoms and laboratory findings. As used herein, the terms “sputa sample” or “sputum sample” refer to a sample of sputum that contains a sufficient amount of sputa to test for the presence of bacterial or other microbial pathogens that are associated with a pulmonary disease or condition. As used herein, the term “saliva sample” relates to a sample predominantly containing saliva. A saliva sample may contain small amounts of sputum, but the sputa content is below that which is optimal for detection of pathogens present in sputa from patients suffering from a pulmonary disease or condition.

The diagnostic system involves a three-step process whereby a sputa sample is obtained from a patient complaining of discomfort and possibly showing signs of a pulmonary disease or condition. A test strip or stick assay specific to the detection of leukocyte esterase or protease activity is contacted with the sputa sample suspected to contain sputum. The test strip assay method for the detection of leukocyte esterase or protease activity is to differentiate sputum from saliva in the sputa sample that had been previously obtained from the patient in need of treatment thereof. Test strip assays are typically designed to produce a color change that can be seen with the naked eye to indicate that a particular analyte activity (here it is leukocyte esterase or protease) has been detected. A positive test result indicating that leukocyte esterase or protease activity has been detected, also indicates that the sputa sample obtained from the patient includes a sufficient amount of sputum to be further analyzed to determine the presence or absence of a specific pathogen associated with a pulmonary infection or condition. This last step is usually done by a clinical laboratory that specifically tests for pathogens associated with pulmonary infectionss and conditions.

In the method of the present invention, a sample of a patient's bodily fluids preferably sputa, is analyzed for leukocyte esterase or protease activity. The sputa sample originates from the respiratory tract, preferably from the lungs, and should contain a sufficient amount of sputum for analysis to determine the presence or absence of bacterial pathogens associated with the lungs. However, since it is estimated that one out of every three samples do not contain a significant amount of sputa for testing purposes, the test strip or stick assays, or other similar assay devices, of the present invention are utilized to solve this major problem by detecting a requisite amount of sputum in a sputa sample. Ideally, the patient should either extract a sample of sputum originating from the lungs via the oral cavity. Once the sputum has entered into the patient's oral cavity it is deposited into a clean receptacle. A satisfactory sputa sample contains a sufficient amount of sputum originating from the lungs so that a reagent test strip can detect the presence of leukocyte esterase or protease activity. Naturally, the sputa sample may contain an amount of saliva. The presence of saliva is not a problem so long as there is a detectable amount of sputum. The sputa sample is then contacted with the reagent test strip. The procedure generally takes less than sixty seconds. However, immersion time periods vary depending of the reagents used in the test strips.

The method of the instant invention involves testing sputa that may contain both sputum and saliva. Reagent test strips manufactured for testing bodily fluids are commercially available (Bayer Corporation) and are all are incorporated by reference herein with the present invention for differentiating a predominantly sputum sample from a predominantly saliva sample. Although the following assays are used for urinalysis, they can be utilized with the present invention: AMES REAGENT STRIPS for Urinalysis are available in bottles of 100 strips: MULTISTIX® 10 SG (#2300A); MULTISTIX® 9 (#2301A); MULTISTIX® 9 SG (#2303A); MULTISTIX® 8 SG (#2304A); MULTISTIX® 7 (#2305A); N-MULTISTIX® SG (#2740A); MULTISTIX® SG (#2741A); N-MULTISTIX® (2829A); MULTISTIX® (#2820A); and BILI-LABSTIX® (#2814A).

Any of these or other commercially available reagent test strips which detect leukocyte esterase or protease activity can be used according to this disclosure to differentiate sputum from saliva. Thus, in a fashion completely analogous to that described above for the Ames REAGENT STRIPS, commercially available reagent test strips produced by Boehringer Mannheim Corporation (now Roche) may be used or adapted for this purpose. For example CHEMSTRIP 9, Catalog No. 417109, provides a readout for leukocytes, and several other analytes. The information provided in the package insert for the CHEMSTRIP 6, 7, 8, 9, 10 (which also provides a readout for specific gravity), is largely analogous to the information provided hereinabove from the Multistix® product. In our hands, testing of sputa samples using the Boehringer product yielded results which, according to this invention, are similar to those obtained using the Multistix® product. Slight adjustments in the color readouts and values thereof may be needed due to the differences between the color charts used by the two manufacturers, but, based on the instant disclosure, those skilled in the art are able to make any necessary adjustments.

Chemical Principles of the Procedure:

Leukocytes: Granulocytic leukocytes contain esterases (or similar enzyme activity) that catalyze the hydrolysis of the derivatized pyrrole amino acid ester to liberate 3-hydroxy-5-phenyl pyrrole. This pyrrole then reacts with a diazonium salt to produce a purple product. The intensity of the purple color developed is used to assign a value to esterase activity as described above.

Reagents (Based on Dry Weight at Time of Impregnation):

Leukocytes: 0.4% w/w derivatized pyrrole amino acid ester; 0.2% w/w diazonium salt; 40.9% w/w buffer; 58.5% w/w nonreactive ingredients.

Leukocytes: Elevated glucose concentrations (≧3 g/dl) or high specific gravity may cause decreased test results. The presence of cephalexin (Keflex®), cephalothin (Keflin®), or high concentrations of oxalic acid may also cause decreased test results. Tetracycline may cause decreased reactivity, and high levels of the drug may cause a false negative reaction.

Leukocytes: Normal saliva samples and nasal secretion will generally yield negative results; positive results (small or greater) are clinically significant. Individually observed trace results may be of questionable clinical significance; however, trace results observed repeatedly may be clinically significant. Positive and repeated trace results indicate the need for further testing of the patient and/or sputa samples, according to medically accepted procedures.

Those skilled in the art will recognize that other chemical components may be used for carrying out the method disclosed herein.

Recommended Procedures for Handling Reagent Strips: All unused strips must remain in the original bottle. Transfer to any other container may cause reagent strips to deteriorate and become unreactive. Do not remove desiccant(s) from bottle. Do not remove strip from the bottle until immediately before it is to be used for testing. Replace cap immediately and tightly after removing reagent strip. Do not touch test areas of the reagent strip. Work areas and specimen containers should be free of detergents and other contaminating substances. Dip test in sputa samples completely, but briefly, to avoid dissolving out the reagents. If using strips visually, read test results carefully at the times specified, in a good light (such as fluorescent) and with the test area held near the appropriate Color Chart on the bottle label. Do not read the strips in direct sunlight. If the strips are used instrumentally, carefully follow the directions given in the appropriate instrument operating manual. Protection against ambient moisture, light and heat is essential to guard against altered reagent.

Discoloration or darkening of reagent areas may indicate deterioration. If this is evident, or if test results are questionable or inconsistent with expected finding, the following steps are recommended: (1) confirm that the product is within the expiration date shown on the label; (2) check performance against known positive control materials (e.g., CHEK-STIX® Control Strips); (3) retest with fresh product.

For best results, performance of reagent strips should be confirmed by testing known negative and positive specimens or controls whenever a new bottle is first opened. Negative and positive specimens or controls may also be randomly hidden in each batch of specimens tested. Each laboratory should establish its own goals for adequate standards of performance, and should question handling and testing procedures if these standards are not met.

Specific Performance Characteristics: Specific performance characteristics are based on clinical and analytical studies. In clinical specimens, the sensitivity depends upon several factors: the variability of color perception; the presence or absence of inhibitory factors, the specific gravity, and the pH; and the lighting conditions when the product is read visually. Because the color of each reagent area changes as the analyte concentration increases, the percentage of specimens detected as positive will increase with the analyte concentration.

Although any test strip or stick assay that detects the presence of the leukocyte esterase analyte, the present invention preferably utilizes test strips (Product Code 5122) by Serim™ or the MULTISTIX® reagent strips by Bayer Corporation®. Leukocyte esterase or protease activity in pulmonary diseases or conditions increases when leukocyte counts increase in response to pulmonary infections typically caused by bacterial pathogens. SERIM™ test strips are designed to give a semi-quantitative indication of the level of leukocytes present in a peritoneal dialysate effluent, thereby providing a presumptive indication of peritonitis. However, the diagnostic system of the present invention can utilize the indicative qualities of these test strips to diagnose a different disease or infection through a different bodily fluid and different method of collecting the bodily fluid thereof.

The chemical principles of some of the test strips or sticks used in the present invention include, but are not limited to, by the reaction of a phenyl pyrrole ester (or an indoxyl ester). In the reaction, esterase in granulocytic leukocytes catalyzes the hydrolysis of a modified pyrrole amino acid ester to yield 3-hydroxy-5-phenyl pyrrole. The released 3-hydroxy-5-phenyl pyrrole then reacts with diazonium salt to produce a purple color (shown in the chemical reaction below).

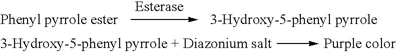

The practical detection limit is usually 10-25 leukocytes/μL. Normal ranges in a urine sample are less than 10 leukocytes/μL, Borderline ranges are 10-20 leukocytes/μL, and pathological ranges are above 20 leukocytes/μL.

### EXAMPLE 1

The presence of one or more of the following conditions are commonly used for diagnosis of a pulmonary disease or condition for the purpose of the present system. After diagnosing the possibility of a pulmonary disease, infection or condition, (generally the patient will complain of chest pain, and shortness of breath and exhibit coughing and fever) the following two exemplary methods are used to obtain a satisfactory sputa sample for further testing and analysis:

Dip Method

1. The patient provides to the physician or medical technician at least one sample of sputum originating from the lungs via the nasal cavity or the oral cavity which is deposited into a clean receptacle.

2. Remove one test strip assay specific for detecting the presence of leukocyte esterase activity from the bottle and immediately replace the cap.

3. Completely immerse the indicator pad of the test strip into the sample, remove immediately and start a timer (time period of immersion depends on the test strip assay utilized with the present invention).

4. Place the test strip (indicator pad facing up) on a flat, clean surface.

5. After immersion of the test strip and after the wait time period has expired for the test strip, immediately compare the color of the indicator pad to the color chart on the bottle or color chart sheet.

Accurate timing is essential to provide optimal results. The reagent strips must be kept in the bottle with the cap tightly closed to maintain reagent reactivity.

Results of Test Strip Method

Generally, most commercially available test strip assays specific in detecting the presence of leukocyte esterase activity are also designed to give a semi-quantitative indication of the level of leukocytes present based on a specific bodily fluid such as urine, saliva, and blood based on in the color of the reacted indicator pad. Although current test strip assays are not designed for sputa samples, the present method can incorporate the test strips to the diagnostic system of the present invention.

Preferably, leukocyte esterase activity is detected by providing a chromogenic reagent on the strip that changes color to an extent proportional to the amount of leukocyte esterase present in the sputa sample. Normal saliva samples without sputum will yield negative results; the indicator pad will match the “Negative” color block. (If the color of the indicator pad is between “Negative” and “Trace”, the results should be considered negative). Not only intact but also already lysed leukocytes are detected. “Trace”, “Small”, or “Large” results indicate significant leukocyte esterase activity and the sputa sample contains a sufficient amount of sputum and should be tested further according to medically accepted procedures for diagnosis of a pulmonary disease or condition. Results with BAYER REAGENT STRIPS for leukocyte esterase activity are obtained in clinically meaningful units directly from the Color Chart comparison when using strips visually. With instrumental use, the reagent pads are “read” by the instrument and the results are displayed or printed. The color blocks and instrumental display values represent nominal values; actual values will vary around the nominal values. Each color block or instrumental display value represents a range of values. Because of specimen and reading variability, specimens with analyte concentrations that fall between nominal levels may give results at either level. Exact agreement between visual results and instrumental results might not be found because of the inherent differences between the perception of the human eye and the optical system of the instruments.

Depending on the product being used, (for example, BAYER REAGENT STRIPS provide tests for leukocyte esterase activity), a user of these strips should refer to the carton and bottle label for specific reagent areas on the product being used. The reagent test areas are ready to use upon removal from the bottle and the entire reagent strip is disposable. The strips may be read visually, requiring no additional laboratory equipment for testing. These reagent strips provide an indication of a contacted fluid's leukocyte esterase activity. The use of reagent indicator strips is, however, one of the most easily conducted, inexpensive and rapid methods for achieving this analysis.

In the method of this invention, a sputa sample of a patient is contacted with a reagent test-strip and, based on the presence or absence of leukocyte esterase, it can quickly be determined if the sample contains a sufficient amount of sputum for further clinical testing to determine if a patient is in need of treatment for a pulmonary disease or condition. The diagnostic system of the present invention is to rapidly aid a physician in the diagnosis of a pulmonary disease or condition which is made by a combination of the clinical symptoms described above and conventional laboratory findings known in the art of analyzing sputum. After detecting the presence leukocyte esterase activity in the sputa sample to determine that there is a sufficient amount of sputum contained within, the sample is then sent to a clinical laboratory designed to test for the presence of a variety of pathogens associated with pulmonary infections and conditions. Once the patient is diagnosed with a respiratory infection or condition, the clinician should prescribe the known medications, e.g., a course of antibiotics to the patient depending on the pathogen identified.

This diagnostic system has the potential to supplant much more expensive and invasive clinical procedures. This diagnostic system resolves the misdiagnosis of patients caused by unsatisfactory samples being sent to the clinical laboratories which have led to patients being miss-prescribed antibiotics or other drugs, or not even prescribed antibiotics when needed. The method of utilizing a test strip or stick assay to differentiate a sputa sample from a saliva sample allows medical personnel to accurately and rapidly send sputa samples containing sputum for analysis of pathogens. The test strip assays produce a color change which can be seen by the naked eye to indicate that luekocyte esterase or protease activity is present in the sample. This activity indicates that the sputa sample obtained from the patient contains sputum. Sputum matter is the result of an infection which contains bacterial pathogens that are associated with many varieties of pulmonary diseases or conditions. Without the use of this test strip method of the present invention, one out of every three sputa samples submitted to clinical laboratories for testing are unsatisfactory. This can cause delay in treatment and unnecessary repetitive testing.

In any event, even with the use of a standard, commercially available reagent test strip, all that is required for the method of this invention is that, in addition to contacting the sputa sample with an appropriate reagent test strip to detect leukocyte esterase activity, further clinical findings of the presence of a specific bacterial pathogen associated with a pulmonary disease or condition is necessary. Accordingly, this invention provides a method for rapidly, non-invasively and inexpensively differentiating sputum from saliva to further clinically determine the presence of a bacterial pulmonary infection.

The patient may be any mammal, including an animal or a human. To aid in the differentiation between sputum and saliva samples, there may be some minor amount of overlap between patients having various clinical conditions that may need to be eliminated before implementing the method of the present invention.

It should be understood that various modifications or changes in light thereof will be suggested to persons skilled in the art and are to be included within the spirit and purview of this application and the scope of the appended claims.

What is claimed is:

1. A diagnostic system for detecting the presence of or evaluation of a pulmonary infection or condition in a patient in need of treatment thereof, comprising:
(a) collecting a sample from said patient, wherein said sample is derived from the respiratory tract of said patient;
(b) providing at least one reagent test strip or stick that comprises at least one reactant for determining the presence of leukocyte esterase or protease activity in said sample;
(c) contacting said sample with said at least one reagent test; and
(d) evaluating the presence or absence of leukocyte esterase or protease activity in said sample; whereby said system enables identification of a sputa sample that comprises an adequate amount of sputum suitable for further testing.

2. The diagnostic system according to claim 1, further comprising testing said sputa sample for the presence of pathogens associated with pulmonary diseases or conditions.

3. The diagnostic system according to claim 1, wherein said patient is a human or animal subject.

4. The diagnostic system according to claim 1, wherein the evaluation of the presence or absence of a leukocyte esterase or protease activity is determined colorimetrically.

5. The diagnostic system according to claim 1, wherein said reagent strip or stick testing is implemented by the dip method to evaluate the presence or absence of leukocyte esterase or protease activity.

6. The diagnostic system according to claim 1, wherein said sample is obtained via the oral cavity.

7. The diagnostic system according to claim 1, wherein said sample is obtained from the mouth, throat, nose or lungs, or combinations thereof, of said patient.
